(19) United States
(12) Patent Application Publication
(10) Pub. No.: US 2008/0004344 A1
(43) Pub. Date: Jan. 3, 2008

(54) Novel Salts of Fumaric Acid Monoalkylesters and Their Pharmaceutical Use

(75) Inventors: Henrik Nilsson, Copenhagen (DE); Jens E.T. Andersen, Vedbaek (DE); Bernd W. Mueller, Flintbek (DE); Joseph R. Robinson, Madison, WI (US); Bonna Robinson, Madison, WI (US)

Correspondence Address: DARBY & DARBY P.C., P.O. BOX 770, Church Street Station, New York, NY, 10008-0770, US

(73) Assignee: ADITECH PHARMA AB, Malmoe (SE)

(21) Appl. No.: 11/719,052

(22) PCT Filed: Nov. 9, 2005
(86) PCT No.: PCT/DK05/00717

§ 371 Date: Sep. 7, 2007

(30) Foreign Application Priority Data
Nov. 10, 2004 (DK) PA 2004 01735
Feb. 11, 2005 (DK) PA 2005 00212
Mar. 23, 2005 (DK) PA 2005 00420

(51) Int. Cl. A61K 31/215; A61P 17/06; A61P 25/00; A61P 37/00; C07C 69/60

(52) U.S. Cl. 514/529; 560/190

(57) ABSTRACT

The present invention relates to novel strontium salts of fumaric acid monoalkylesters. The salts are suitable for use as active substances in the treatment of e.g. psoriasis or other hyperproliferative, inflammatory or autoimmune disorders either alone or in combination with another fumaric acid ester

### FIELD OF THE INVENTION

The present invention relates to novel strontium salts of fumaric acid monoalkylesters. The salts are suitable for use as active substances in the treatment of e.g. psoriasis or other hyperproliferative, inflammatory or autoimmune disorders, either alone or in combination with another fumaric acid ester.

### BACKGROUND OF THE INVENTION

Fumaric acid esters, i.e. dimethylfumarate in combination with ethylhydrogenfumarat have been used in the treatment of psoriasis for many years. The combination is marketed under the tradename Fumaderm®. It is in the form of tablets intended for oral use and it is available in two different dosage strengths (Fumaderm® initial and Fumaderm®):

| | Fumaderm ® Initial | Fumaderm ® |
|---|---|---|
| Dimethylfumarate | 30 mg | 120 mg |
| Ethylhydrogenfumarate, | 67 mg | 87 mg |
| calcium salt | | |
| Ethylhydrogenfumarate, | 5 mg | 5 mg |
| Magnesium salt | | |
| Etylhydrogenfumarate, | 3 mg | 3 mg |
| Zinc salt | | |

The two strengths are intended to be applied in an individually based dose regimen starting with Fumaderm® initial in an escalating dose, and then after e.g. three weeks of treatment switching to Fumaderm®. Both Fumaderm® initial and Fumaderm® are enteric coated tablets.

Another marketed composition is Fumaraat 120® containing 120 mg of dimethylfumarate and 95 mg of calcium monoethylfumarate (TioFarma, Oud-Beijerland, Netherlands). In a recent publication (Litjens et al. Br. J. Clin. Pharmacol. 2004, vol. 58:4, pp. 429-432), the pharmacokinetic profile of Fumaraat 120® is described in healthy subjects. The results show that a single oral dose of Fumaraat 120® is followed by a rise in serum monomethylfumarate concentration and only negligible concentrations of dimethylfumarate and fumaric acid is observed. The results indicate that dimethylfumarate is rapidly hydrolyzed to monomethylfumarate in an alkaline environment, but according to the authors not in an acid environment. As the composition is enteric coated, it is contemplated that the uptake of fumarate takes place mainly in the small intestine, where dimethylfumarate before uptake is hydrolysed to the monoester due to an alkaline environment or it may rapidly be converted due to esterases in the circulation. Furthermore, the study shows that tₘₐₓ and Cₘₐₓ are subject to food effect, i.e. tₘₐₓ is prolonged (mean for fasted conditions is 182 min, whereas for fed conditions mean is 361 min) [lag time is 90 min for fasted and 300 min for fed] and Cₘₐₓ is decreased (fasted: 0.84 mg/l, fed: 0.48 mg/l) by concomitant food-intake. Another study (Reddingius W. G. Bioanalysis and Pharmacokinetics of Fumarates in Humans. Dissertation ETH Zurich No. 12199)1997) in healthy subjects with two tablets of Fumaderm® P forte revealed Cₘₐₓ values (determined as monoethyl- or monomethylfumarate) in a range from 1.0 to 2.4 μg/ml and a tₘₐₓ in a range of from 4.8 to 6.0 hours.

U.S. Pat. No. 6,277,882 and U.S. Pat. No. 6,355,676 disclose respectively the use of alkyl hydrogen fumarates and the use of certain fumaric acid mono alkyl ester salts for preparing micro tablets for treating psoriasis, psoriatic arthritis, neurodermatitis and enteritis regionalis Crohn. U.S. Pat. No. 6,509,376 discloses the use of certain dialkyl fumarates for the preparation of pharmaceutical preparations for use in transplantation medicine or the therapy of autoimmune diseases in the form of micro tablets or pellets. U.S. Pat. No. 4,959,389 disclose compositions containing different salts of fumaric acid monoalkyl ester alone or in combination with dialkyl fumarate. The Case report “Treatment of disseminated granuloma annulare with fumaric acid esters” from BMC Dermatology, vol. 2, no.5, 2002, relates to treatment with fumaric acid esters.

However, therapy with fumarates like e.g. Fumaderm® frequently gives rise to gastro-intestinal side effects such as e.g. fullness, diarrhea, upper abdominal cramps, flatulence and nausea.

Furthermore, the present commercially available product contains a combination of two different esters of which one of the esters (namely the ethylhydrogenfumarate which is the monoethylester of fumaric acid) is present in three different salt forms (i.e. the calcium, magnesium and zinc salt). Although each individual form may have its own therapeutic profile it would be advantageous to have a much simpler product, if possible, in order to obtain a suitable therapeutic effect.

Accordingly, there is a need to develop novel drug compounds of therapeutically or prophylactically active fumaric acid esters that provide an alternative and potential improved treatment e.g. with a reduction in gastro-intestinal related side effects upon oral administration.

### SUMMARY OF THE INVENTION

The present invention provides in one aspect new strontium salts of monoalkylesters of fumaric acid. These novel drug compounds are contemplated to lead to an improved treatment of conditions susceptible to fumarate treatment.

The mono- and dimethylester as well as the mono- and diethylester of fumaric acid have a poor solubility in water and this may be a factor leading to a low bioavailability (the bioavailability for the dimethylester and the diethylester of fumaric acid is regarded as very variable after oral administration). Formation of strontium salts (compared to calcium or zink salts) may lead to a more suitable solubility in water or to a more suitable hydrophilic-liphophilic balance and, furthermore, due to the beneficial effect of the strontium ion itself, the novel salts according to the invention are contemplated to lead to an improved treatment regimen.

In further aspects the invention provides use of said new strontium salts of monoalkylesters of fumaric acid in medicine and for combating tissue degenerative processes and more specifically in treatment of conditions such as Psoriasis, Psoriatic arthritis, Neurodermatitis, Inflammatory bowel disease such as Crohn's disease and Ulcerative colitis, autoimmune diseases such as Polyarthritis, Multiple sclerosis (MS), Juvenile-onset diabetes, Hashimoto's thyroiditis, Grave's disease, SLE (systemic lupus erythematosus), Sjögren's syndrome, Pernicious anemia, Chronic active (lupoid) hepatitis, rheumatoid arthritis (RA) and optic neuritis, pain such as radicular pain, pain associated with radiculopathy, neuropathic pain or sciatica/sciatic pain; or for treatment and/or prevention of any of the following conditions: prevention of rejection following organ transplantation; Sarcoidosis; Necrobiosis lipoidica; and/or Granuloma annulare. In another aspect of the invention the use of said new strontium salts of monoalkylesters of fumaric acid for the manufacture of a pharmaceutical composition is provided. In another further aspect pharmaceutical compositions are provided. In yet further aspects methods for preparation of such new salts are provided.

### BRIEF DESCRIPTION OF THE FIGURES

FIG. 1 shows the crystal structure of the di-strontium-tetrakis-(mono methyl fumarate)-tetra hydrate prepared in example 1

FIG. 2 shows the crystal lattice of the di-strontium-tetrakis-(mono methyl fumarate)-tetra hydrate prepared in example 1

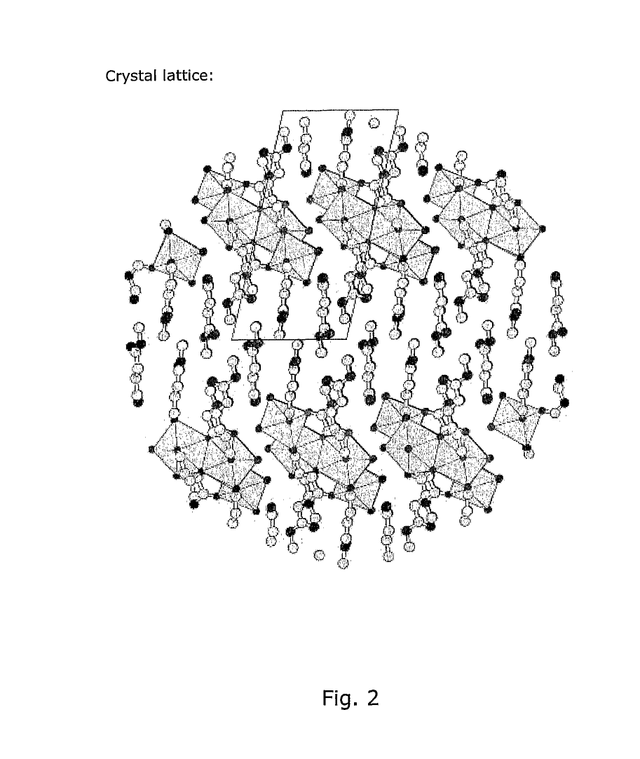

### DISCLOSURE OF THE INVENTION

Accordingly, the present invention relates to novel strontium salts of a mono-(C₁-C₅)alkylester of fumaric acid that may be used alone or in combination treatment e.g. with a di-(C₁-C₅)alkylester of fumaric acid or other active substances.

The term (C₁-C₅)alkyl or C₁₋₅-alkyl refers to a branched or un-branched alkyl group having from one to five carbon atoms inclusive, such as methyl, ethyl, 1-propyl, 2-propyl, 1-butyl, 2-butyl, 2-methyl-2-propyl, 2-methyl-1-propyl and pentyl.

The present invention also provides compositions including controlled release compositions comprising a novel salt according to the invention as well as to the use of the novel salts in medicine. Furthermore, the present invention provides a method for the manufacturing of the novel salts according to the invention.

A composition according to the invention comprising a novel salt may—upon oral administration and in comparison to that obtained after oral administration of Fumaderm® tablets in an equivalent dosage—give a reduction in GI (gastrointestinal) related side-effects.

A suitable way of reducing the gastrointestinal related side effects is likely to be by administration of a novel salt in the form of a controlled release composition.

As used in the present invention, a gastrointestinal (GI) side effect may include, but is not limited to diarrhea, stomach ache, stomach pain, abdominal pain, abdominal cramps, nausea, flatulence, tenesmus, meteorism, an increased frequency of stools, a feeling of fullness and upper abdominal cramps.

In the present context, a reduction of GI related side effects is intended to denote a decrease in severity and/or incidence among a given treated patient population, compared to the GI side effects observed after administration of the composition according to the invention compared with that of Fumaderm®. A reduction in GI related side effects according to this definition could thus be construed as a substantial reduction in incidence of any of the GI side effect listed above, such as at least a 10% reduction in incidence or more preferably at least 20% reduction in incidence or even more preferable a more than 30% reduction in incidence. A reduction in GI related side effect can also be expressed as a substantial reduction in severity in any of the GI side effects listed above, such as a reduction in severity and/or frequency of diarrhea, stomach ache, stomach pain, abdominal pain, abdominal cramps, nausea, flatulence, tenesmus, meteorism, increased frequency of stools, a feeling of fullness or upper abdominal cramps. The reduction of GI related side effects, as described above, can be monitored in a clinical trial setting, either comparing the administration of the composition according to the invention head on with Fumaderm® or with placebo. In case of a placebo controlled trial, the incidence of GI related side effects in the patients receiving the composition according to the invention compared to the placebo group, can be compared to historical trials comparing Fumaderm® to placebo (see e.g. Altmeyer et al, J. Am. Acad. Dermatol. 1994; full reference: Altmeyer P J et al, Antipsoriatic effect of fumaric acid derivatives. Results of a multicenter double-blind study in 100 patients. J. Am. Acad. Dermatol. 1994; 30:977-81). Typically, patients suffering from psoriasis are included in such a study, and typically more than 10% of the body surface area will be affected by psoriasis (severe psoriasis). However, patients in whom between 2 and 10 percent of the body surface area is affected can also be included (moderate psoriasis). Patients can also be selected based on the Psoriasis Area Severity Index (PASI). Typically, patients within a certain range of PASI are included, such as between 10 and 40, or such as between 12 and 30, or such as between 15 and 25. Patients with any type of psoriasis may be included (chronic plaque type, exanthematic guttate type, pustular type, psoriatic erythroderma or palmoplantar type), but in some cases only patients with the chronic plaque type are included. About 15 to 20 patients in each treatment group (composition according to the invention and Fumaderm® or placebo) are sufficient in most cases, but more preferably about 30 to 50 patients are included in each arm of the study. Total study duration can be as short as one day to one week, but more preferably the study will run for 8 weeks to 12 weeks or up to 16 weeks. The side effects can e.g. be assessed as the total number of times a certain side effect was reported in each group (irrespective of how many patients have experienced the side effect), or the side effects can be assessed as the number of patients that have experienced a certain side effect a certain number of times, such as at least once or at least twice or at least three times during the duration of the study. Furthermore, the severity of a side effect can be monitored, or a certain severity of a side effect can be required for it to qualify as a side effect in the study. A convenient way of assessing the severity of a side effect is via a visual analogue (VAS) scale.

Active Substance

In one aspect of the invention, the active substance in a composition of the invention is a strontium salt of a fumaric acid monoester selected from the group consisting of monomethylfumarate, monoethylfumarate, monopropylfumarate, monobutylfumarate and monopentylfumarate. The active substance may be used in combination with another active fumaric acid ester such as a dialkylfumarate like e.g. dimethylfumarate, diethylfumarate, dipropylfumarate, dibutylfumarate, dipentylfumarate, methyl-ethylfumarate, methyl-propylfumarate, methyl-butylfumarate or methyl-pentylfumarate, or monoalkylfumarates such as monomethylfumarate, monoethylfumarate, monopropylfumarate, monobutylfumarate or monopentylfumarate including pharmaceutically acceptable salts thereof.

In a specific embodiment of the invention, the fumaric acid ester is a mono-(C₁-C₅)alkylester of fumaric acid that is present in the form of a strontium salt. Accordingly, the present invention relates in a further aspect to the following novel compounds:

Strontium salt of

monomethylester of fumaric acid (strontium bis-monomethyl fumarate)

monoethylester of fumaric acid (strontium bis-monoethyl fumarate)

monopropylester of fumaric acid (strontium bis-monopropyl fumarate)

monobutylester of fumaric acid (strontium bis-monobutyl fumarate)

monopentylester of fumaric acid (strontium bis-monopentyl fumarate).

In yet an embodiment according to the invention, a strontium salt of the monomethylester of fumaric acid is provided.

In another embodiment, a composition according to the invention comprises a strontium salt of a mono(C₁-C₅)alkylester of fumaric acid together with a di(C₁-C₅)alkylester of fumaric acid (e.g. dimethylfumarate) as the active substances.

In a further embodiment, the composition according to the invention comprises as active substances a combination of a strontium salt of a mono(C₁-C₅)alkylester of fumaric acid and a mono(C₁-C₅)alkylester of fumaric acid (e.g. monomethylfumarate) optionally In the form of a pharmaceutically acceptable salt like e.g. its sodium, potassium, calcium, magnesium and/or zinc salt.

In yet a further embodiment, the strontium salt according to the invention is in crystalline form.

Strontium

Strontium is found naturally exclusively as a non-radioactive stable element. The rationale for selecting strontium as a counter ion to fumaric acid esters is according to the inventors due to strontium's beneficial therapeutic effect e.g. on pain (see e.g. WO 03/28742), and strontium's potential beneficial effect in reducing the risk of developing gastritis (Meunier P J et al, The effects of strontium ranelate on the risk of vertebral fracture in women with postmenopausal osteoporosis, N Engl J Med. Jan. 29, 2004;350(5):459-68), a condition similar to some of the side effects observed with Fumaderm®.

Synthesis of strontium salts of fumaric acid monoesters According to the Invention

Fumaric acid, its monomethyl ester and its dimethylester are well known compounds that may be isolated from plants or synthesized. The synthesis of the monomethyl ester of fumaric acid is not necessarily straightforward because of symmetry. Accordingly, attempts to synthesize the monomethyl ester by adding methanol to fumaric acid may inevitably lead to formation of the dimethyl ester. In addition, the synthesis may be complicated by the presence of the double bond, which under elevated temperature and pressure may hydrolyse and produce oxalic acid.

The terms “alkaline condition”, “neutral condition” and “acid condition” refer to conditions wherein the pH is above approximately 7, approximately 7 and below approximately 7, respectively.

Synthesis and purification of strontium salts of organic acids is influenced by ambient carbon dioxide that is a constituent of laboratory water. In alkaline solution, carbon dioxide is adsorbed from the air by formation of carbonate:
CO₂(g)+2OH⁻(aq)→CO₃²⁻(aq)+H₂O(l) (1)

When strontium monomethyl fumarate is dissolved in water, the pH-value of the solution raises to alkaline values (pH˜9-10), owing to production of hydroxyl ions:
Sr(C₅H₅O₄)₂+2H₂O→Sr²⁺(aq)+2C₅H₆O₄(aq)+2OH⁻(aq) (2)

Thus, the production of carbonate by eq. 1 is promoted by dissolution of the strontium salt. The carbonate then reacts with the mixture to form strontium carbonate that is insoluble.
Sr²⁺(aq)+CO₃²⁻(aq)→SrCO₃(s) (3)

Thus, upon recrystallisation, the purity of the strontium metallorganic salt decreases, owing to consumption of strontium from the precipitation of strontium carbonate (eq. 3).

Further, decarboxylation reactions commence by raising the temperature of solutions containing strontium carboxylates.

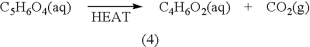

Synthesis of strontium carboxylates in high yield may therefore be obstructed by several unfavourable side reactions. Accordingly, in one embodiment of the invention, synthesis of a strontium salt is performed by de-aerated water, preferably by nitrogen, Ar or another inert gas.

The sodium salt of the fumaric acid is commercially available (e.g. from Alfa Aesar or Sigma-Aldrich). Synthesis of the monomethyl ester of fumaric acid has been described by Zhong et al. [Synthesis of mold inhibitor monomethyl fumarate. Jingxi Huagong (2002), 19(6), 343-345], as shown in Scheme 1, and is commercially available from Sanmenxia xiawei chemical co., ltd., China. To the best of the inventors' knowledge, neither strontium salts of monoalkylesters of fumaric acid nor methods for their production/synthesis have been described before.

### Scheme 1. Synthesis of monomethyl fumarate from maleic acid and methanol

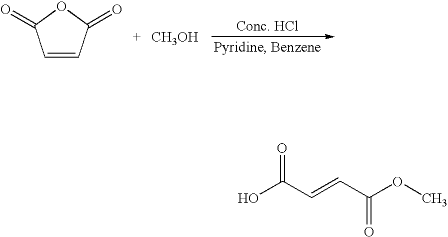

Molar ratio maleic anhydride/MeOH=1:1

Reaction temperature=60° C.

Reaction time=3 hours

Quantity of HCl=6%

Quantity of pyridine=2%

Expected yield=83%

Several ways of producing the strontium salts of fumaric acid monoalkylesters according to the invention may be contemplated.

In an embodiment of the invention, a method for preparing a strontium salt according to the invention is provided, comprising formation of a dialkyl ester of fumaric acid followed by precipitation by either strontium hydroxide or by strontium chloride in acid solution:

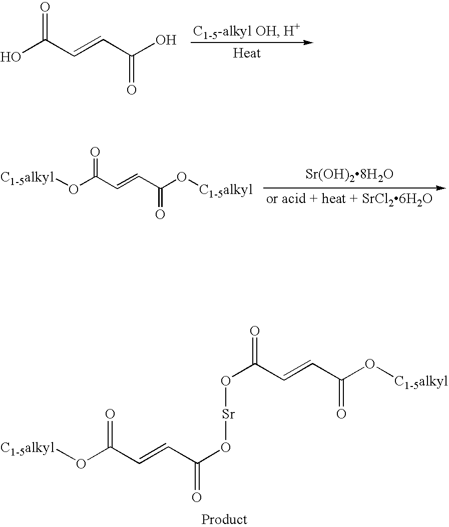

In another embodiment of the invention, a method for preparing a strontium salt according to the invention is provided, comprising formation of fumaric anhydride by a sulphuryl method followed by hydrolysis in the presence of strontium chloride:

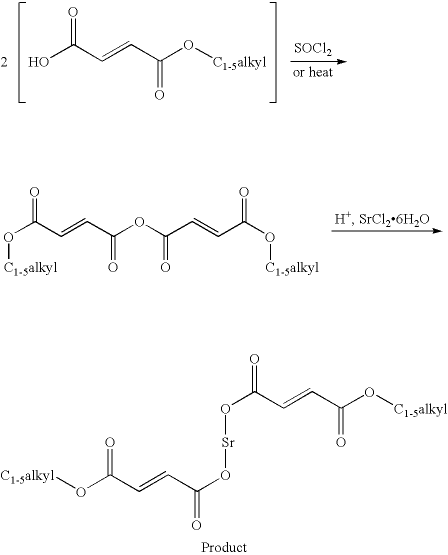

In yet another embodiment of the invention, a method for preparing a strontium salt according to the invention is provided, comprising reacting the disodium salt of fumaric acid and alkanol in acid solution:

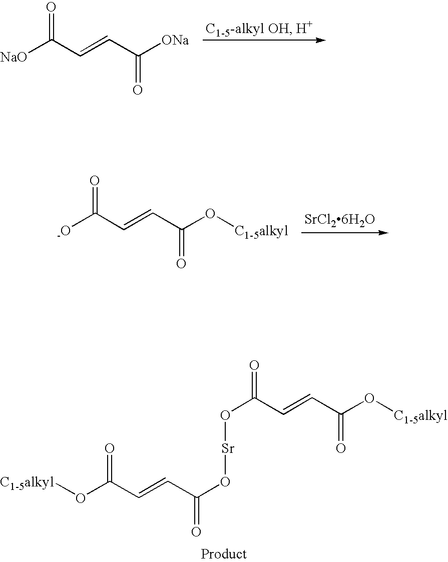

In yet another embodiment of the invention, a method for preparing a strontium salt according to the invention is provided, comprising formation of bis-monoalkylfumarate from the monoalkyl ester of fumaric acid and precipitation under acid, alkaline or neutral conditions in the presence of strontium chloride:

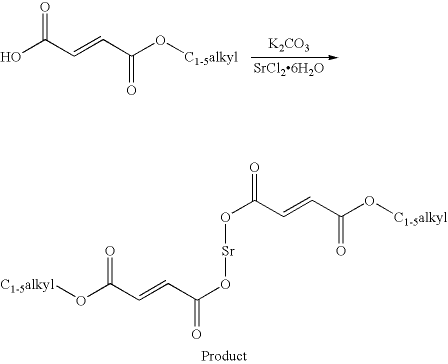

In a further embodiment of the invention, a method for preparing a strontium salt according to the invention is provided, comprising reaction of strontium carbonate with monoalkylfumarate:

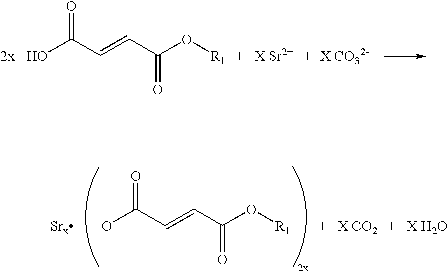

wherein x denotes the stoichiometric factor and R₁ is any alkyl group with the number of carbon atoms ranging between 1 to 5. The structure may crystallise with an unknown number of water molecules, which is subsequently determined experimentally after synthesis.

In yet a further embodiment of the invention, a method for preparing a strontium salt according to the invention is provided, comprising formation of strontium monoalkylfumarate by reaction of strontium hydroxide with monoalkylfumarate dissolved in water.

Synthesis of strontium bis-monomethylfumarate in Accordance with the Invention

A total of six pathways are contemplated below for the production of the desired compound, strontium bis-monomethylfumarate. In order to prevent formation of strontium carbonate and to prevent decarboxylation, it is contemplated that all synthesis may be performed without access to air and under pressurised conditions.

1. Precipitation in Acid Solution

Synthesis of bis-monomethylfumarate by synthesis of the dimethyl ester of fumaric acid followed by precipitation by either strontium hydroxide or by strontium chloride in acid solution (Scheme 2). It is contemplated that the precipitation in acid solution will be possible to perform with a relatively high yield, while it is contemplated that the alkaline method will require high concentration and temperature, which poses a risk of hydrolysis. In addition, synthesis of the monomethyl ester from the dimethyl ester will require much optimisation and special conditions, similar to those of scheme 1.

### Scheme 2. Synthesis of bis-monomethylfumarate by Formation of dimethyl fumarate Followed by Precipitation by Either strontium hydroxide or by strontium chloride in acid Solution

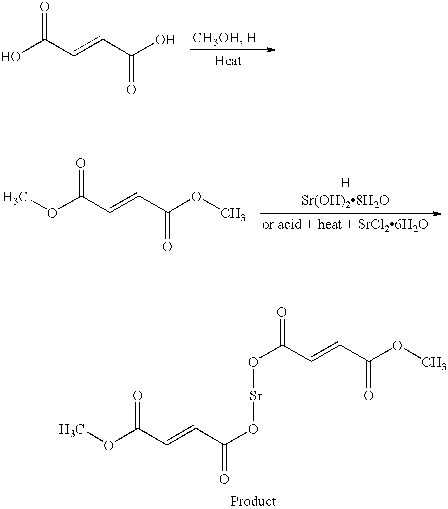

2. Sulphuryl Method Followed by Hydrolysis

Synthesis of bis-monomethylfumarate from fumaric acid and formation of fumaric anhydride by the sulphuryl method followed by controlled hydrolysis in the presence of strontium chloride (Scheme 3). It is contemplated that this method produces small amounts of the desired product, and at the same time there will be a risk of recovering high amounts of the fumaric acid reagent. To avoid this, careful monitoring of the pH-value and of the temperature must be observed. Formation of the anhydride may also be accomplished by heating solid fumaric acid, which then avoids handling of poisonous sulphuryl chloride.

### Scheme 3. Synthesis of bis-monomethylfumarate from fumaric acid and Formation of fumaric anhydride by the sulphuryl Method Followed by Controlled hydrolysis in the Presence of strontium chloride

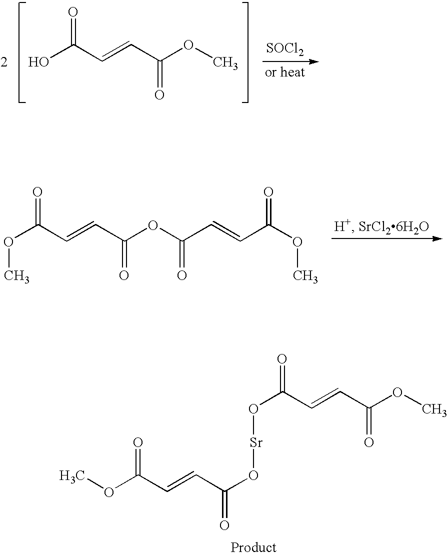

3. Disodium salt as Starting Material

This method resembles method No. 1. Synthesis of bis-monomethylfumarate from the disodium salt of fumaric acid and methanol in acid solution (Scheme. 4). It is contemplated that this synthesis produces a mixture of monomethyl esters and of dimethyl esters and the synthesis procedure must therefore be optimised to increase the yield of the monomethyl ester. By raising the pH-value by dilution, it is contemplated that addition of strontium chloride will produce some amounts of the product.

### Scheme 4. Synthesis of bis-monomethylfumarate from the disodium salt of fumaric acid and methanol in acid Solution

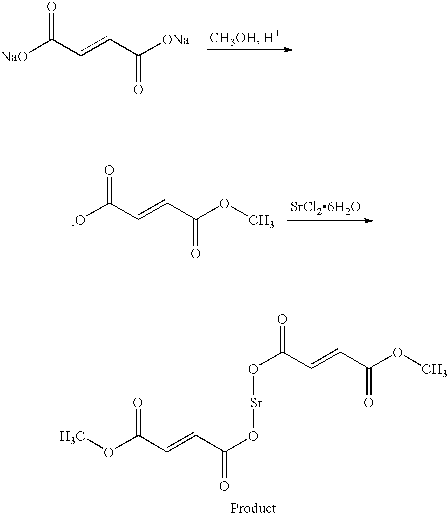

4. Monomethyl ester of fumaric acid as Starting Material

Synthesis of bis-monomethylfumarate from the monomethyl ester of fumaric acid and precipitation under acid, alkaline or neutral conditions in the presence of strontium chloride (Scheme. 5). It is contemplated that this method will produce the product in high yield. Furthermore, it is also contemplated that one could use strontium hydroxide instead of strontium chloride and potassium carbonate.

### Scheme 5. Synthesis of bis-monomethylfumarate from the monomethyl ester of fumaric acid and Precipitation Under acid, alkaline or neutral Conditions in the Presence of strontium chloride

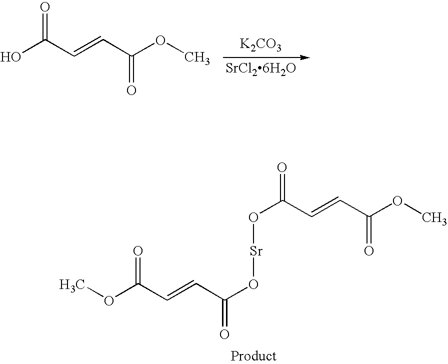

5. Monomethyl ester of fumaric acid and strontium carbonate as Starting Material

This method corresponds to the synthesis of pathway 4 above (“Monomethyl ester of fumaric acid as starting material”). Strontium carbonate is added by sprinkling the powder over a solution of dissolved monomethylfumarate (Scheme 6). Vigorous evolution of gaseous carbon dioxide occurs during the reaction. In order to avoid hydrolysis of the monomethylfumarate, the reaction is performed at low temperature (room temperature or below) and the pH-value of the solution is kept within the acid range of pH-values. This procedure is contemplated to provide the product in high yield and high purity.

### Scheme 6. Synthesis of di-strontium-tetrakis-(monomethyl fumarate)-tetra-hydrate by Reaction of strontium carbonate with monomethylfumarate. The Reaction Proceeds at Room Temperature

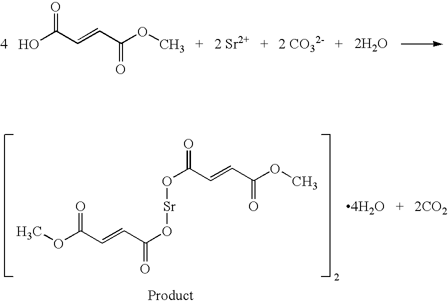

6. Reaction of strontium hydroxide with monomethylfumarate Dissolved in Water.

It is contemplated that addition of strontium hydroxide to a solution of monomethylfumarate will produce the desired product in high yield and high purity (Scheme 7). In order to avoid hydrolysis of the monomethylfumarate, the reaction is performed at low temperature (room temperature or below) and the pH-value of the solution is kept within the acid range of pH-values.

### Scheme 7. Synthesis of strontium monomethyl fumarate by Reaction of strontium hydroxide with monomethylfumarate Dissolved in Water. The Reaction Proceeds at Room Temperature

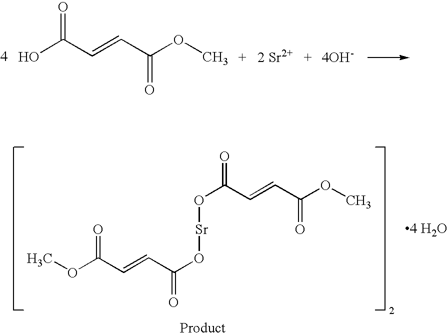

Although the above-given schemes are directed to the manufacturing of the strontium salt of fumaric acid monomethylester, a person skilled in the art is able to use the information in order to substitute the starting materials and reagents employed in order to obtain strontium salts of other fumaric acid monoalkylesters.

Dosage

In one aspect of the invention, the dosage of a compound according to the invention such as a strontium-monomethylfumarate to be administered should provide a peak plasma concentration (Cₘₐₓ) of monomethylfumarate in a range of from about 0.4 to about 2.0 mg l⁻¹ after a single dose administration to humans.

In another aspect of the invention, the dosage of a compound according to the invention such as strontium-monomethylfumarate to be administered should provide an area under the plasma concentration vs. time profile (AUC_{0-∞}) of from about 30 to 150 mg×min l⁻¹ after a single dose administration to humans.

In another aspect of the invention, the total daily dosage of a compound according to the invention such as strontium-monomethylfumarate to be used should provide a clinical effect as measured by a reduction in the PASI score similar to or better than what is obtained after administration of Fumaderm® or Panaclar® at a total daily dose equivalent to 1080 mg dimethylfumarate.

In another aspect of the invention, the total daily dosage of a compound according to the invention such as strontium-monomethylfumarate to be used should provide a clinical effect as measured by a reduction in the PASI score similar to or better than what is obtained after administration of Fumaderm® or Panaclar® at a total daily dose equivalent to 720 mg dimethylfumarate (120 mg given as two tablets three times daily).

In another aspect of the invention, the total daily dosage of a compound according to the invention such as strontium-monomethylfumarate to be used should provide a clinical effect as measured by a reduction in the PASI score similar to or better than what is obtained after administration of Fumaderm® or Panaclar® at a total daily dose equivalent to 480 mg dimethylfumarate.

In another aspect of the invention, the total daily dosage of a compound according to the invention such as strontium-monomethylfumarate to be used should provide a clinical effect as measured by a reduction in the PASI score similar to or better than what is obtained after administration of Fumaderm® or Panaclar® at a total daily dose equivalent to 360 mg dimethylfumarate (120 mg given as one tablet three times daily).

In another aspect of the invention, the total daily dosage of a compound according to the invention such as strontium-monomethylfumarate to be used should provide a clinical effect as measured by a reduction in the PASI score similar to or better than what is obtained after administration of Fumaderm® or Panaclar® at a total daily dose equivalent to 240 mg dimethylfumarate.

The clinical effect of the compounds according to the invention may be measured in a double-blind, placebo controlled, parallel-group study. Eligible patients for testing for the effect on e.g. psoriasis are patients who have had psoriasis (chronic, exanthematic guttate, erythrodermic, plamoplantar, or pustular) for at least 1 year. Patients should typically have a baseline PASI of 16-24. Systemic treatment should be discontinued 4 weeks before study initiation. Topical treatment should be discontinued 2 week before study initiation. Only topical salicylic acids and emollients should be allowed during the study period.

Patients should be randomised to either the placebo-group or to a group receiving the pharmaceutical composition according to the invention with a daily dosage of monomethylfumarate. The total number of patients to be included will depend on the specific study-design but may be e.g. 80 patients with 40 patients on placebo and 40 patients on active treatment.

The treatment period is 12-16 weeks. The primary measure of efficacy is the reduction in PASI score between baseline and at the end of treatment.

The present compounds, compositions and kits are contemplated to be suitable to use in the treatment of one or more of the following conditions:
a. Psoriasis
b. Psoriatic arthritis
c. Neurodermatitis
d. Inflammatory bowel disease, such as
   i. Crohn's disease
   ii. Ulcerative colitis
e. autoimmune diseases:
   i. Polyarthritis
   ii. Multiple sclerosis (MS)
   iii. Juvenile-onset diabetes mellitus
   iv. Hashimoto's thyroiditis
   v. Grave's disease
   vi. SLE (systemic lupus erythematosus)
   vii. Sjögren's syndrome
   viii. Pernicious anemia
   ix. Chronic active (lupoid) hepatitis
   x. Rheumatoid arthritis (RA)
   xi. Optic neuritis
f. Pain such as radicular pain, pain associated with radiculopathy, neuropathic pain or sciatica/sciatic pain
g. Organ transplantation (prevention of rejection)
h. Sarcoidosis
i. Necrobiosis lipoidica
j. Granuloma annulare

It is also contemplated that other counter ions than strontium may be suitable for use in the treatment of conditions susceptible to fumaric acid alkylesters. Suitable counter ions may be positively charged metal ions or organic compounds comprising e.g. an amino functional group.

Psoriasis has been proposed to potentially be associated with Crohn's disease (Najarian D J, Gottlieb A B, Connections between psoriasis and Crohn's disease. J Am Acad Dermatol. June 2003; 48(6):805-21), celiac disease (Ojetti V et al, High prevalence of celiac disease in psoriasis. Am J Gastroenterol. November 2003;98(11):2574-5.), psychiatric or psychological disease, such as depression or a life crisis (Gupta M A, Gupta A K, Psychiatric and psychological co-morbidity in patients with dermatologic disorders: epidemiology and management. Am J Clin Dermatol. 2003;4(12):833-42. and Mallbris L et al, Psoriasis phenotype at disease onset: clinical characterization of 400 adult cases. J Invest Dermatol. March 2005;124(3):499-504.), overweight, diabetes mellitus, excess consumption of alcohol/alcoholism, as well as psoriatic arthritis.

The present invention thus relates in one aspect to a method of treating psoriasis, psoriatic arthritis, neurodermatitis, inflammatory bowel disease, such as Crohn's disease and ulcerative colitis, autoimmune diseases, such as polyarthritis, multiple sclerosis (MS), juvenile-onset diabetes mellitus, Hashimoto's thyroiditis, Grave's disease, SLE (systemic lupus erythematosus), Sjögren's syndrome, Pernicious anemia, Chronic active (lupoid) hepatitis, Rheumatoid arthritis (RA) and optic neuritis, pain such as radicular pain, pain associated with radiculopathy, neuropathic pain or sciatica/sciatic pain, organ transplantation (prevention of rejection), sarcoidosis, necrobiosis lipoidica or granuloma annulare, which method comprises administering orally to a patient in need thereof, an effective dosage of a compound according the invention.

The present invention relates in another aspect to the use of a compound according to the invention for the preparation of a medicament for the treatment of psoriasis, psoriatic arthritis, neurodermatitis, inflammatory bowel disease, such as Crohn's disease and ulcerative colitis, autoimmune diseases, such as polyarthritis, multiple sclerosis (MS), juvenile-onset diabetes mellitus, Hashimoto's thyroiditis, Grave's disease, SLE (systemic lupus erythematosus), Sjögren's syndrome, Pernicious anemia, Chronic active (lupoid) hepatitis, Rheumatoid arthritis (RA) and optic neuritis, pain such as radicular pain, pain associated with radiculopathy, neuropathic pain or sciatica/sciatic pain, organ transplantation (prevention of rejection), sarcoidosis, necrobiosis lipoidica or granuloma annulare.

In one aspect of the invention, a compound according to the invention for use in the treatment of one or more conditions, where the condition is selected from psoriasis, psoriatic arthritis, neurodermatitis and multiple sclerosis (MS), is provided. In yet a further aspect of the invention, a compound according to the invention for use in the treatment of psoriasis, is provided.

Furthermore, the invention also relates to treating an individual suffering from one of the conditions in the abovementioned lists, more specifically psoriasis or psoriatic arthritis, with a compound, composition or kit according to the invention, said individual further being in treatment with

a) a topical anti-psoriatic drug such as 1) vitamin D or derivatives thereof (calcipotriol, calcipotriene), 2) a corticosteroid (such as e.g. betamethasone, desoximethasone, fluocinolone, momethasone, hydrocortisone aceponate, fluticasone, clobethasol, clobethasone, hydrocortisone butyrate, desonide, triamcinolone or hydrocortisone), 3) tazaroten, 4) ditranol, 5) tacrolimus (FK-506) and other calcineurin inhibitors, such as pimecrolimus or 6) any combination of 1-5 and/or

b) an oral anti-psoriatic drug such as 1) an oral retinoid (such as acitretin or etretinate) combined or not combined with PUVA, 2) cyclosporine and other calcineurin inhibitors, such as ISA247, tacrolimus and pimecrolimus, 3) methotrexate, 4) hydroxyurea, 5) azathioprine, 6) sulphasalazine, 7) a fumarate derivative (such as e.g. Fumaderm or BG-12), 8) rosiglitazone (Avandia) and other peroxisome proliferator-activated-γ (PPARγ) agonists or modulators, such as pioglitazone, farglitazar, GW1929, GW7845, MC-555, MBX-102/MBX-10, MBX-1828, MBX-2044, CLX-0921, R-483, reglitazar, naveglitazar (LY-519818/LY-818), netoglitazone (MCC-555), CS-7017, troglitazone, ciglitazone, tesaglitazar, isaglitazone, balaglitazone, muraglitazar, TAK-654, LBM642, DRF 4158, EML 4156, T-174, TY-51501, TY-12780, VDO-52 or AMG-131(T131) or any combination of 1-8 and/or

c) a parenterally administered anti-psoriatic drug such as 1) alefacept (Amevive), 2) etanercept (Enbrel), 3) efalizumab (Raptiva), 4) onercept, 5) adalimumab (Humira) or any combination of 1-5 and/or

d) an inhibitor of TNF-α not mentioned in the list under section c) above (e.g. CDP 870 or infliximab (Remicade)), administered via an enteral or parenteral route and/or

e) tisocalicitrate and/or NCX 1022 and/or IDEC-131 and/or MEDI-507, and/or

f) An NSAID or a COX or a LOX inhibitor such as e.g. a COX-2 inhibitor or a COX/5-LOX inhibitor, and/or

g) an anti-diabetic or anti-obesity drug, such as biguanides such as metformin; metformin XR; a sulphonylurea such as chlorpropamide, glipizide, gliclazide, glyburide/glibenclamide or glimepiride; Glucovance (metformin+glyburide); Metaglip (glipizide+metformin); a peroxisome proliferator-activated-γ (PPARγ) agonist or modulator, such as rosiglitazone (Avandia), pioglitazone, farglitazar, GW1929, GW7845, MC-555, MBX-102/MBX-10, MBX-1828, MBX-2044, CLX-0921, R-483, reglitazar, naveglitazar (LY-519818/LY-818), netoglitazone (MCC-555), CS-7017, troglitazone, ciglitazone, tesaglitazar, isaglitazone, balaglitazone, muraglitazar, TAK-654, LBM642, DRF 4158, EML 4156, T-174, TY-51501, TY-12780, VDO-52 or AMG-131(T131); Avandamet (rosiglitazone+metformin); Actos (pioglitazone+metformin); Avandaryl (rosiglitazone maleate+glimepiride); a benzoimidazole such as FK-614; CS-917; TA-1095; ONO-5129; TAK-559; TAK-677/AJ-9667; a d-phenylalanine inducer such as senaglinide; c-3347; NBI-6024; ingliforib; BVT 3498; LY 929; SGLT2 inhibitors; CS 011; BIM 51077; R1438; R1439; R1440; R1498; R1499; AVE 0847; AVE 2268; AVE 5688; AVE 8134; TA-6666; AZD 6370; SSR 162369; TLK-17411; NN 2501; MK 431; KGA-2727; MK-767; CS-872; a beta-3 receptor antagonist such as N-5984; an alpha-glucosidase inhibitor such as acarbose, voglibose or miglitol; a glinitide/meglitinide analogue or carbamoylmethylbensoeic acid derivative such as mitiglinide, repaglinide or nateglinide; a DPP-IV inhibitor such as LAF 237 (vildagliptin), DPP728, P93/01, P32/98, PT-630 or saxagliptin; GLP-1 or GLP-1 analogues, such as exenatide, Exenatide-LAR, liraglutide (NN 2211), ZP 10/AVE 0010, LY 307161, betatropin, CJC-1131, GTP-010, SUN E7001 or AZM 134; pramlinitide acetate; insulin or insulin analogues, such as Humalog (insulin lispro), Humulin, Novolin, Novolog/NovoRapid (insulin aspart), Apidra (insulin glulisine), Lantus (insulin glargine), Exubera, Levemir/NN 304 (insulin detemir), AERx/NN 1998, Insuman, Pulmonary insulin or NN 344; sibutramine or other blockers of the presynaptic reuptake of serotonin and noradrenalin; orlistat and other inhibitors of GI lipases; β3-adrenergic receptor agonists; uncoupling proteins; (specific) antagonists of PPARγ (Peroxisome Proliferator-Activated Receptor γ); insulin secretagogues; rimonabant and other CB1 endocannabinoid receptor antagonists; bupropion; topiramate; leptin agonists; ciliary neurotrophic factor; peptide analogues of the human growth hormone fragment 177-191; cholecystokinin-A receptor agonists; melanocortin-3 agonists; noradrenergic drugs such as phentermine, diethylpropion, phendimetrazine or benzphetamine; or any combination of the anti-diabetic or anti-obesity drugs mentioned above, and/or

h) a drug potentially useful in the treatment of substance abuse e.g. alcohol abuse such as naltrexone, acamprosate, disulphiram or Vivitrex (naltrexone long acting injection), and/or,

i) a drug potentially useful in the treatment of Crohn's disease such as
1. 5-ASA compounds such as sulfasalazine, oral 5-ASA formulations or rectal 5-ASA formulations,
2. glucocorticosteroids such as systemic steroids (e.g. budesonide or prednisolone) or topically acting steroids (e.g. budesonide),
3. antibiotics such as metronidazole or quinolones (e.g. ciprofloxacine, ofloxacine, norfloxacine, levofloxacine or moxifloxacine),
4. immunosuppressives such as azathioprine, 6-mercaptopurine or methotrexate,
5. nutritional therapies such as elemental or polymeric formulas or pre- and probiotics,
6. biological therapies e.g. TNF-α inhibitors such as infliximab, adalimumab, CDP870, CDP571, etanercept or onercept,
7. symptomatic agents such as anti-diarrheals or anti-spasmodics.

Examples of suitable NSAIDs are piroxicam, diclofenac, nabumetone, propionic acids including naproxen, flurbiprofen, fenoprofen, ketoprofen and ibuprofen, fenamates including mefenamic acid, paracetamol, indomethacin, sulindac, meloxicam, apazone, pyrazolones including phenylbutazone, salicylates including aspirin.

Examples of suitable COX-2 inhibitors are rofecoxib (Vioxx), valdecoxib (Bextra), celecoxib (Celebrex), etoricoxib (Arcoxia), lumiracoxib (Prexige), parecoxib (Dynastat), deracoxib (Deram), tiracoxib, meloxicam, nimesolide, (1,1-dimethylheptyl)-6a,7,10,10a-tetrahydro-l-hydroxy-6,6dimethyl-6H-dibenzo[b,d]pyran carboxylic acid (CT-3), 2(5H)-Furanone, 5,5-dimethyl (l-methylethoxy) [4(methylsulfonyl)phenyl]-(DFP); Carprofen (RIMADYL), (Acetyloxy)-benzoic acid, 3-[(nitrooxy)methyliphenyl ester (NCX4016), P54 (CAS Reg. No. 130996 0) 2,6-Bis(1,1-dimethylethyl) [(E)-(2-ethyl-1,1-dioxo isothiazolidinylidene)methyl]phenol (S-2474), 5(R)-Thio sulfonamide-3(2H)-benzofuranone (SVT-2016) and N-[3-(Fonnyl-amino)oxophenoxy-4Hbenzopyranyl]methanesulfonamide (“T-614”); or a pharmaceutically acceptable salt thereof.

Examples of suitable COX/5-LOX inhibitors are licofelone (ML-3000 or [2,2-dimethyl-6-(4-chlorophenyl)-7-phenyl-2,3,dihydro-1H-pyrrolizine-5-yl]-acetic acid), di-tert-butylphenols, such as (E)-(5)-(3,5-di-tert-butyl-4-hydroxybenzylidence)-2-ethyl-1,2-isothiazolidine-1,1-dioxide (S-2474), darbufelone or tebufelone and pharmacologically active metabolites as well as derivatives such as dihydro-dimethyl-benzofuran and PGV-20229, dihydro-dimethyl-benzofuran, thiophene derived compounds such as RWJ-63556, N-hydroxy-N-methyl-4-(2,3-bis-(4-methoxyphenyl)-thiophen-5-yl)-butanamide (519812), methoxytetrahydropyran derivatives, oxygenated xanthones such as 1,3,6,7-Tetrahydroxyxanthone (norathyriol)-pyrazole thiocarbamates, pyrazoles such as modified forms of phenidone containing compounds or the tri-flouro-benzole substituted pyrazoline derivative BW-755C, tepoxaline and derivatives and di-tert-butylpyrimidines.

It is contemplated that such combination therapy leads to an improved therapeutic response and/or an increased convenience for the individual, compared to said individual being treated without the compound, composition or kit according to the invention.

In a further aspect, the invention relates to a method of reducing side effects associated with oral treatment of any of the conditions a-j listed above, in which method the active pharmaceutical ingredient for treating said condition is used in combination with one or more of the following agents:

a) an antacid such as 1) magnesium hydroxide, 2) magnesium trisilicate, 3) aluminium hydroxyde gel, 3) sodium hydrogencarbonate, 4) magaldrat or any combination of 1-5 and/or

b) a histamine H-2 antagonist such as 1) cimetidine, 2) ranitidine, 3) nizatidine, 4) famotidine, 5) roxatidine, 6) lafutadine or any combination of 1-6 and/or

c) a cytoprotective agent such as 1) sucralfate, 2) tripotassium dictitratobismuthate, 3) carbenoxolone, 4) prostaglandin E-2 analogues such as misoprostol, 5) ecabet, 6) cetraxate HCl, 7) teprenone, 8) troxipide, 9) dicyclomine hydrochloride, 10) sofalcon or any combination of 1-10 and/or

d) a proton pump inhibitor (PPI) such as 1) omeprazole, 2) esomeprazole, 3) lansoproazole, 4) pantoprazole, 5) rabeprazole, 6) CS-526/R-105266, 7) AZD 0865, 8) soraprazan or any combination of 1-8, and/or

e) an NSAID or a COX or a LOX inhibitor such as e.g. a COX-2 inhibitor or a COX/5-LOX inhibitor, and/or

f) pentoxifylline, e.g. at a dose range of from 400 to 800 mg/day.

Cosmetic and/or Pharmaceutical Compositions

The novel salts of the invention may be presented in the form of a cosmetic or pharmaceutical composition.

The salts according to the invention may be used for preparing pharmaceutical compositions for oral administration in the form of micro-pellets, micro-tablets, capsules (such as soft and hard gelatine capsules), granulates and tablets such as e.g. described in U.S. Pat. No. 6,509,376 or U.S. Pat. No. 6,355,676 incorporated herein by reference. Further suitable pharmaceutical compositions are preparations for cutaneous and transdermal administration in the form of ointments, plasters, lotions or shower preparations and for parenteral administration in the form of aqueous micro-dispersions, oil-in-water emulsions or oily solutions for rectal administration of suppositories or micro-enemas.

In specific embodiments, the invention relates to a pharmaceutical composition that may be administered one, two or more times daily, such as once or twice or three times daily. Examples of such compositions are e.g. compositions in the form of solid dosages forms (e.g. tablets, capsules, pellets, beads etc.) that are coated with an enteric coating.

The novel salts may also solve or reduce the problems related to the appearance of gastro-intestinal side-effects upon oral administration of fumaric acid esters. Furthermore, by prolonging and/or delaying the release of the active substance from the composition it is envisaged that the local concentration of the active substance at specific sites of the gastro-intestinal tract is reduced (compared with that of Fumaderm®) which in turn leads to a reduction in gastrointestinal side-effects. Accordingly, compositions that enable a prolonged and/or a slow release of a fumaric acid ester as defined above are within the scope of the present invention.

Such compositions are well-known to the skilled artisan and include e.g. diffusion-controlled drug delivery systems, osmotic pressure controlled drug delivery systems, erodible drug delivery systems etc. Moreover, there are pharmaceutical companies that based on a specific technology (such as mentioned above) can provide a specific composition with specific release characteristics of the active substance. Accordingly, a person skilled in the art will know how to obtain a suitable product once he has realized a specific need in respect of a particular drug substance. By way of example, Eurand is one of such companies that offer technical solutions in order to obtain a controlled release pharmaceutical composition containing a specific active substance and having specific requirements with respect to the release of the active substance from the composition (see e.g. http://www.eurand.com). Another company is MacroMed, Inc. that has developed a technology involving a so-called SQZgel™ (http://macromed.com, SQZgel™'s mechanism of action is a pH-sensitive polymer mixture combined with an outer coating. In the acidic environment of the stomach the polymer imbibes with water and swells, entrapping the drug. Upon entering the higher pH of the intestines, the polymer slowly shrinks, or “squeezes” at a “dialed-in” rate releasing the active composition in a sustained manner.), or Egalet a/s that has a specific extrusion based technology (http://www.egalet.com, Key elements of the Egalet® technology are a biodegradable coat and a matrix, comprising the active drug, which is surface erodible, hydrophobic and composed of PEG-stearate. One of the Egalet® technologies is the 2K Egalet® constant release system, which is a 2-component production model consisting of coat and matrix. The drug is evenly distributed throughout the Egalet® matrix for constant release over time). These and other technologies like e.g. the Eurand technologies Diffucaps (Drug release profiles are created by layering active drug onto a neutral core such as sugar spheres, crystals or granules followed by a rate-controlling, functional membrane. Diffucaps/Surecaps beads are small in size, approximately 1 mm or less in diameter. By incorporating beads of differing drug release profiles into hard gelatin capsules, combination release profiles can be achieved.), Diffutabs (The Diffutab technology incorporates a blend of hydrophilic polymers that control drug release through diffusion and erosion of a matrix tablet.), Minitabs (Eurand Minitabs are tiny (2 mm×2 mm) tablets containing gel-forming excipients that control drug release rate. Additional membranes may be added to further control release rate.), Orbexa (This technology produces beads that are of controlled size and density with a defined-based granulation extrusion and spheronization techniques. The resultant beads can be coated with release rate controlling membranes for additional release rate control and may be filled into capsules or provided in sachet form.) and SDS (Eurand's SDS technology uses functional polymers or a combination of functional polymers and specific additives, such as composite polymeric materials, to deliver a drug to a site of optimal absorption along the intestinal tract. In order to achieve this, Eurand first produces multiparticulate dosage forms such as Diffucaps or Eurand Minitabs, which incorporate the active drug. These dosage forms are then coated with pH dependent/independent polymeric membranes that will deliver the drug to the desired site. These are then filled into hard gelatin capsules.) are also of interest in the present context.

An interesting technology for use in formulating compositions according to the present invention is the so-called MeltDose® technology as described in WO 03/004001 (see http://www.lifecyclepharma.com. MeltDose® involves formulating solubilized, individual molecules into tablets. By formulating individual molecules, the primary limitation of oral absorption of drugs with low water-solubility is removed, and a superior bioavailability can be attained.). By employing this technology it is possible to obtain a particulate material that is suitable for processing into various pharmaceutical dosage forms e.g. in the form of pellets or tablets. Furthermore, the technology is suitable for use as it is possible to obtain a suitable release profile of the active substance, e.g. such as those release profiles described herein. In one embodiment, pellets suitable for use may have a mean particle size larger than 2000 μm. In another embodiment, pellets suitable for use may have a mean particle size of from about 0.01 μm to about 250 μm.

Another specific suitable formulation principle for use in the present context is formulation in a lipophilic environment such as, e.g., soft gelatin capsules. Vegicaps Soft from Scherer (a soft capsule technology based on carrageenan and starch. While this new dosage form is 100% plant-derived, it still offers all the key attributes of traditional soft gelatin capsules. These include a soft and flexible dosage form that provides ease of swallowing.) is a suitable example of such a formulation principle (please refer to http://www.rpscherer.de/page.php?pageID=94).

A further specific example of a suitable formulation comprises a compound according to the invention together with vitamin E concentrate in soft or hard gelatin capsules. This formulation, in a modified form, is the basis of the commercial cyclosporine product, Neoral®, containing, among other things, corn oil-mono-di-triglycerides, polyoxyl 40 hydrogenated castor oil NF, DL-α-tocopherol USP (part of the vitamin E family), gelatin NF, glycerol, iron oxide black, propylene glycol USP, titanium dioxide USP, carmine, and alcohol in addition to cyclosporine.

Another specific example of a suitable formulation comprises a compound according to the invention together with ethanol, tocopherolethylene glycol 1000 succinate (TPGS), corn oil and wax in soft or hard gelatin capsules. This product can be a semi-solid or solid dosage form. The release rate of this formulation is dependent on degradation due to lipases in the intestine.

A further example of a suitable formulation comprises the formulation of a compound according to the invention together with ethanol, tocopherolethylene glycol 1000 succinate (TPGS), corn oil and polyglycolized glycerides (e.g. Gelucire) in soft or hard gelatin capsules. This product can be a semi-solid or solid dosage form. The release rate of this formulation is dependent on degradation due to lipases in the intestine.

A further example of a suitable formulation is an oral pulsed dose drug delivery system. This dosage form can be perceived as a modified form of the Schering Repetab tablets. A portion of the composition of the present invention is put in the core of a tablet.

The core can for example be made by conventional wet granulation or continuous granulation such as extrusion followed by compaction of the granulate into tablets. The core is then coated using an appropriate technology, preferably by airsuspension using an enteric coating polymer such as Eudragits.

The first releasing dose is compression coated on the core or air-suspension coated either with the enteric coat or on top of the enteric coat. In a embodiment of the invention, the first releasing dose is air-suspension coated with the enteric coat. In a further embodiment of the invention, the first releasing dose is compression coated on the core, in order to avoid release of the composition according to the invention prior to the degradation of the enteric coat, such degradation typically occurring at pH values higher than those found in the gastric ventricle; i.e. the degradation of the enteric coat typically occurs after passage of the gastric ventricle.

A further example of a suitable formulation is an oral sustained drug delivery system. A portion of the composition of the present invention is put in the core of a tablet.

The core can for example be made by conventional wet granulation or continuous granulation such as extrusion followed by compaction of the granulate into tablets. The core is coated using an appropriate technology, preferably by airsuspension using ethylcellulose and a hydrophilic excipient such as hydroxyl propyl cellulose (HPC).

The first releasing dose is compression coated on the core or air-suspension coated either with the enteric coat or on top of the enteric coat. In a preferred embodiment of the invention, the first releasing dose is air-suspension coated with the enteric coat. In a further embodiment of the invention, the first releasing dose is compression coated on the core, in order to avoid release of the composition according to the invention prior to the degradation of the enteric coat, such degradation typically occurring at pH values higher than those found in the gastric ventricle; i.e. the degradation of the enteric coat typically occurs after passage of the gastric ventricle.

A further example of a suitable formulation is obtained via crystal engineering, such as e.g. described in WO 03/080034, which is hereby incorporated by reference.

Accordingly, in another embodiment the composition of the invention comprises the novel salt in the form of micro-crystals with hydrophilic surfaces. Furthermore, in another embodiment of the invention, the micro-crystals are filmcoated directly, in order to achieve a sustained release formulation.

Another specific example of a suitable formulation comprises complexation of the compound according to the present invention with genuine cyclodextrins and cyclodextrin-derivatives (e.g. alkyl- and hydroxyalkyl-derivatives or sulfobutyl-derivatives). The complexation is achieved in accordance with well known methods. It is contemplated that such a complexation leads to a higher solubility and a higher dissolution rate of the composition according to the invention, compared to the composition prior to complexation. Furthermore, it is contemplated that such a complexation leads to a higher bioavailability of the composition according to the invention, compared to the composition prior to complexation.

In specific embodiments, the invention relates to a controlled release pharmaceutical composition that may be administered one, two or more times daily, such as once or twice or three times daily. Furthermore, the composition may be designed so that it releases the fumaric acid ester relatively independent on pH, i.e. the release is not dependent on pH in the gastrointestinal tract. Examples of such compositions are e.g. compositions in the form of solid dosages forms (e.g. tablets, capsules, pellets, beads etc.) that are coated with a controlled release coating. Suitable materials for controlled release coatings are e.g. cellulose and cellulose derivatives including methylcellulose, ethylcellulose and cellulose acetate, or poly(ethylene-co-vinyl acetate), poly (vinyl chloride).

The release of the fumaric acid ester typically takes place in three steps from a composition coated with a diffusion controlled membrane:

i) firstly, water (from the GI tract) diffuses into the dosage form from the surroundings,

ii) secondly, at least some of the fumaric acid ester present in the dosage form dissolves by the action of water,

iii) the dissolved fumaric acid ester diffuses out of the dosage form and into the surroundings (i.e. the GI tract)

Other examples include e.g. matrix tablets or dosage form containing a multiplicity of units each in the form of a matrix system. The active substance is embedded in a matrix containing e.g. cellulose and cellulose derivatives including microcrystalline cellulose, hydroxypropyl methyl cellulose, hydroxypropyl cellulose and methylcellulose, povidone, poly(ethyleneoxide) (PEO), polyethylene glycol (PEG), poly (vinyl alcohol) (PVA), xanthan gum, carrageenan and other synthetic materials. Substances normally used as pharmaceutically acceptable excipients or additives may be added to a matrix composition.

Other examples of suitable compositions are e.g. hydrogels, i.e. monolithic systems wherein the active substance is embedded in a water-swellable network polymer. Materials suitable for use include e.g. hydrophilic vinyl and acrylic polymers, polysaccharides like alginates, and poly(ethylene oxide).

In specific embodiments, a composition according to the invention has a pH controlled release (also known as a pH dependent release) of the fumaric acid ester. Normally, the release is designed so that only a small amount, if any, of the fumaric acid ester is released in the stomach (pH up to about 3), whereas the fumaric acid ester is released in the intestines (pH shifts to about 6-7). Such a pH controlled release can be obtained by providing a composition of the invention with an enteric coating (the whole composition or, if the composition is a multiparticulate composition, the individual units) or by providing a composition that releases the fumaric acid by a pH-dependent osmotic mechanism, or by employment of suitable enzymes.

Examples of suitable substances for use as enteric coating materials include polyacrylamides, phthalate derivatives such as acid phthalates of carbohydrates, amylose acetate phthalate, cellulose acetate phthalate, other cellulose ester phthalates, cellulose ether phthalates, hydroxypropylcellulose phthalate, hydroxypropylethylcellulose phthalate, hydroxypropylmethylcellulose phthalate, methylcellulose phthalate, polyvinyl acetate phthalate, poly acrylic methacrylic acid copolymers, shellac and vinyl acetate and crotonic acid copolymers, etc.

The compositions mentioned above having a pH independent release may also be formulated to release the fumaric acid ester e.g. by providing the composition with an outer layer of an enteric coating.

Furthermore, the compositions may be formulated in such a manner that an initial delay in release of the fumaric acid ester is obtained. Such a delay may be obtained e.g. by choosing an outermost coating that in a time-controlled manner degrades (e.g. erodes) and only when this outermost coating is eroded away, the release of the fumaric acid ester starts.

In the present context, a controlled release composition is a composition that is designed to release the fumaric acid ester in a prolonged, slow and/or delayed manner compared to the release of the commercially available product Fumaderm®, when tested under comparable conditions (e.g. for in vivo studies: dose equivalents, with or without standardized meal etc., or for in vitro studies: dose equivalents, dissolution test apparatus and working conditions including e.g. composition, volume and temperature of dissolution medium employed, rotation speed etc.).

The release in vivo may be tested by measuring the plasma concentration at predetermined time periods and thereby obtaining a plasma concentration versus time profile for the fumaric acid ester in question or, if relevant, a metabolite thereof. Furthermore, it is contemplated that metabolism already takes place within the gastrointestinal tract or during passage of the gastro-intestinal mucosa, or upon first passage through the hepatic circulation. Accordingly, when dimethylfumarate is administered, the relevant component to search for in the plasma is the monomethyl ester and not the dimethylester of fumaric acid. In addition, in the case when strontium-monomethylfumarate is administered, the relevant component to measure in plasma is the monomethylfumarate.

Apart from providing pharmaceutical compositions having different content of the compounds according to the invention, the invention in one aspect also provides kits containing two or more containers e.g. with compositions having various amounts of the compounds according to the invention included. Such kits are e.g suitable for use in those situations where an increasing dosage is required over time.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims. Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges and are encompassed within the invention, subject to any specifically excluded limit in the stated range. Where the stated range includes one or both of the limits, ranges excluding either or both of those included limits are also included in the invention. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are described. All publications mentioned herein are incorporated herein by reference to disclose and describe the methods and/or materials in connection with which the publications are cited. It must be noted that as used herein and in the appended claims, the singular forms “a”, “an”, and “the” include plural referents unless the context clearly dictates otherwise. The patents and publications discussed herein are provided solely for their disclosure prior to the filing date of the present application. Nothing herein is to be construed as an admission that the present invention is not entitled to antedate such patent or publication by virtue of prior invention. Further, the dates of publication provided may be different from the actual publication dates which may need to be independently confirmed. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. The figures shown herein are not necessarily drawn to scale, with some components and features being exaggerated for clarity.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

### EXAMPLE 1

### Synthesis of di-strontium-tetrakis-(mono methyl fumarate)-tetra hydrate

Chemicals

Monomethyl fumarate (C₅H₆O₄): Aldrich 97%, CAS 2756-87-8, FW 130.10, mp. 143-147° C.

Strontium carbonate (SrCO₃): Aldrich 99.9%, CAS 1633-05-2, FW 147.63, d=3.7

Solute: Distilled water.

Procedure

In a measuring glass of volume 250 mL, 250 mL of distilled water is added at 10° C. Monomethyl fumarate (2.000 g) is added while stirring by a glass rod. Strontium carbonate (1.135 g) is added to the solution in one portion and the measuring glass is covered by parafilm. The solution is allowed to rest overnight and then suction filtered by a Büchner funnel. The production exhibits medium solubility and the total yield is between 25-80% after the first step of filtration. More product can be obtained at lower purity by evaporation of the solvent.

Preparation of crystals for single-crystal x-ray crystallography is performed by adding small volumes of the stock solution to a crystallisation beaker. Crystals are formed within a few days by storing the beaker in an oven at 45° C.

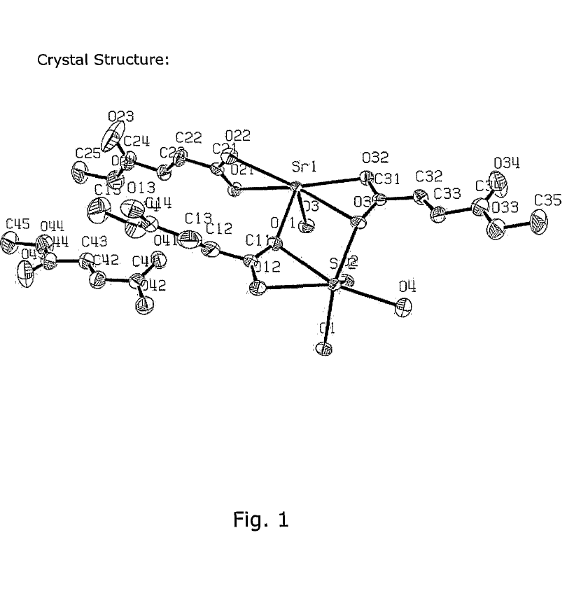

The result of the x-ray analysis shows that the product contains four units of monomethyl fumarate and two strontium ions. The unit cell contains four water molecules. Thus, the formula weight of the product is determined as 76.64 g/mol. The reaction equation is shown below. The crystal structure and molecular lattice of the crystal are shown in FIG. 1.

No melting point of the product is found. Upon heating, the product disintegrates at temperatures above 200° C.

Reaction Equation:

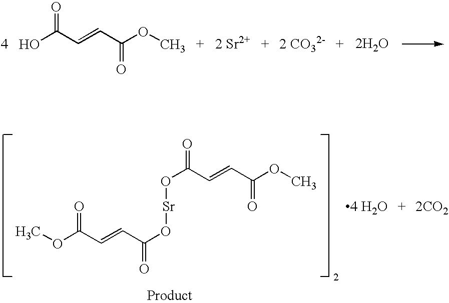

### EXAMPLE 2

### In Vitro Analysis of Immune-Modulatory Effects of strontium monomethylfumarate

PBMC were isolated from blood of two healthy volunteers (female, age: 28 years) via Ficoll density gradient centrifugation. The purified cells were used in parallel cultures for monocyte stimulations using Lipopolysaccharide (LPS) and T cell specific stimulations using bead-coupled antibodies directed against the T cell activation antigens CD3 and CD28.

The following monocyte stimulation cultures were set up in 1 ml tissue culture medium at 1×10⁶ cells per well of a 24-well plate: (i) unstimulated cells, (ii) control, i.e. LPS-stimulated cells (LPS at 1 ng/ml), (iii) LPS-stimulated cells (LPS at 1 ng/ml) in the presence of 8 concentrations of strontium MMF (equivalent to MMF ranging from 3 mM to 1 μM, in half-logarithmic dilutions), and (iv) unstimulated cells in the presence of 8 concentrations of strontium MMF (MMF ranging from 3 mM to 1 μM, in half-logarithmic dilutions).

The following T cell specific stimulation cultures were set up in 1 ml tissue culture medium at 1×10⁶ cells per well of a 24-well plate: (i) unstimulated cells, (ii) control, i.e. CD3/CD28-stimulated cells, (iii) CD3/CD28-stimulated cells in the presence of 8 concentrations of strontium MMF (equivalent MMF ranging from 3 mM to 1 μM, in half-logarithmic dilutions). Cultures were incubated in a tissue culture incubator at 37° C., 5% CO₂ for 20 h, culture supernatants were harvested at the end of the culture period and used for cytokine quantitation. The measured cytokines in cell culture supernatants from either monocyte stimulation cultures (IL-8, IL-10, TNF-α) or T cell stimulation cultures (IL-2, IL-4) were quantitated with ELISAs (Becton Dickinson) according to the manufacturers instructions. The resulting optical density (OD) was measured on a Packard Fusion Reader and OD values were translated to cytokine concentrations (in pg/ml) with the use of the Fusion Reader Data Analysis Software.

TABLE 1
Cytokines secreted through T cell stimulation

| | | | Donor 1 | | Donor 2 | |
|---|---|---|---|---|---|---|
| | Sample | | Conc (SD) pg/ml | | Conc (SD) pg/ml | |
| IL-2 | Unstimulated | | 29 | (47) | 1 | (10) |
| | CD3/CD28 bds | | 939 | (22) | 226 | (11) |
| CD3/CD28 | 3 | mM MMF | 1 | (7) | 3 | (4) |
| beads | 1 | mM MMF | 0 | (0) | 1 | (4) |
| | 300 | μM MMF | 2 | (5) | 0 | (0) |
| | 100 | μM MMF | 170 | (20) | 65 | (22) |
| | 30 | μM MMF | 4592 | (229) | 2079 | (17) |
| | 10 | μM MMF | 5901 | (232) | 1905 | (13) |
| | 3 | μM MMF | 4699 | (308) | 1754 | (203) |
| | 1 | μM MMF | 5787 | (104) | 2559 | (68) |
| IL-4 | Unstimulated | | 0 | (0) | 0 | (0) |
| | CD3/CD28 bds | | 21 | (2) | 2 | (1) |
| CD3/CD28 | 3 | mM MMF | 0 | (0) | 0 | (0) |
| beads | 1 | mM MMF | 0 | (0) | 0 | (0) |
| | 300 | μM MMF | 0 | (0) | 0 | (0) |
| | 100 | μM MMF | 3 | (1) | 0 | (0) |
| | 30 | μM MMF | 92 | (4) | 20 | (0) |
| | 10 | μM MMF | 69 | (2) | 12 | (1) |
| | 3 | μM MMF | 70 | (2) | 10 | (1) |
| | 1 | μM MMF | 60 | (1) | 12 | (1) |

TABLE 2
Cytokines secreted through monocyte stimulation

| | | | Donor 1 | | Donor 2 | |
|---|---|---|---|---|---|---|
| | Sample | | Conc (SD) pg/ml | | Conc (SD) pg/ml | |
| TNF-alpha | Unstimulated | | 19 | (1) | 7 | (2) |
| | LPS 1 ng/ml | | 502 | (32) | 360 | (10) |
| LPS | 3 | mM MMF | 0 | (0) | 0 | (0) |
| 1 ng/ml | 1 | mM MMF | 0 | (0) | 11 | (2) |
| | 300 | μM MMF | 20 | (9) | 96 | (8) |
| | 100 | μM MMF | 250 | (5) | 711 | (48) |
| | 30 | μM MMF | 1787 | (14) | 3289 | (36) |
| | 10 | μM MMF | 1624 | (16) | 2559 | (24) |
| | 3 | μM MMF | 631 | (10) | 650 | (17) |
| | 1 | μM MMF | 464 | (4) | 459 | (19) |
| | 3 | mM MMF | 0 | (0) | 0 | (0) |
| | 1 | mM MMF | 0 | (0) | 3 | (1) |
| | 300 | μM MMF | 3 | (4) | 12 | (3) |
| | 100 | μM MMF | 313 | (3) | 236 | (23) |
| | 30 | μM MMF | 1343 | (10) | 682 | (15) |
| | 10 | μM MMF | 570 | (14) | 47 | (1) |
| | 3 | μM MMF | 38 | (2) | 10 | (1) |
| | 1 | μM MMF | 29 | (1) | 13 | (3) |
| IL-10 | Unstimulated | | 24 | (14) | 11 | (4) |
| | LPS 1 ng/mL | | 759 | (41) | 1083 | (45) |
| | 3 | mM MMF | 0 | (0) | 0 | (0) |
| | 1 | mM MMF | 0 | (0) | 0 | (0) |
| | 300 | μM MMF | 0 | (0) | 0 | (0) |
| | 100 | μM MMF | 7 | (11) | 40 | (39) |
| | 30 | μM MMF | 351 | (59) | 800 | (61) |
| | 10 | μM MMF | 1026 | (28) | 2035 | (103) |
| | 3 | μM MMF | 886 | (11) | 1385 | (67) |
| | 1 | μM MMF | 775 | (13) | 1125 | (25) |
| LPS | 3 | mM MMF | 1 | (1) | 0 | (0) |
| 1 ng/ml | 1 | mM MMF | 2 | (4) | 0 | (0) |
| | 300 | μM MMF | 0 | (0) | 0 | (0) |
| LPS | 100 | μM MMF | 7 | (6) | 0 | (0) |
| 1 ng/ml | 30 | μM MMF | 62 | (2) | 32 | (9) |
| | 10 | μM MMF | 106 | (4) | 13 | (2) |
| | 3 | μM MMF | 13 | (4) | 5 | (1) |
| | 1 | μM MMF | 11 | (2) | 11 | (2) |
| IL-8 | Unstimulated | | 50781 | (1570) | 26289 | (607) |
| | LPS 1 ng/mL | | 481381 | (4505) | 262540 | (16770) |
| | 3 | mM MMF | 176 | (19) | 577 | (69) |
| | 1 | mM MMF | 1696 | (95) | 5363 | (128) |
| | 300 | μM MMF | 12354 | (618) | 31040 | (807) |
| | 100 | μM MMF | 60312 | (1061) | 153980 | (4316) |
| | 30 | μM MMF | 179340 | (8516) | 315447 | (19237) |
| | 10 | μM MMF | 249798 | (10172) | 446267 | (11245) |
| | 3 | μM MMF | 219130 | (2925) | 349450 | (21287) |
| | 1 | μM MMF | 138285 | (1324) | 241352 | (10026) |
| LPS | 3 | mM MMF | 86 | (4) | 424 | (7) |
| 1 ng/ml | 1 | mM MMF | 558 | (38) | 2825 | (23) |
| | 300 | μM MMF | 7135 | (299) | 31370 | (689) |
| | 100 | μM MMF | 80524 | (1761) | 1285660 | (3495) |
| | 30 | μM MMF | 1369127 | (5671) | 1357587 | (2386) |
| | 10 | μM MMF | 1259306 | (20255) | 70691 | |
| | 3 | μM MMF | 26255 | (1093) | 24672 | (237) |
| | 1 | μM MMF | 35264 | (144) | 25146 | (1028) |

The influence of strontium monomethylfumarate on cytokines secreted through monocyte stimulation is shown in Table 1. Strontium MMF induced the pro-inflammatory cytokine TNF-alpha as single stimulant as well as to act co-stimulatory on LPS-stimulated monocytes. Likewise TNF-alpha, the cytokine IL-10 was also shown to be induced and co-induced by strontium MMF. Finally, IL-8 did not induce a co-stimulation on LPS stimulated monocytes. In unstimulated monocytes Strontium MMF did induce an induction of IL-8.

In addition, the influence of strontium MMF on cytokine secreted through T cell stimulation is shown in Table 2. The data suggest that strontium MMF leads to a co-induction of IL-2 and IL-4 after T-cell stimulation.

The current data suggests that strontium MMF is able to influence cytokines of relevance for the treatment of psoriasis and other immunological diseases.

1. A strontium salt of a mono-(C1-C5)alkylester of fumaric acid.

2. The strontium salt according to claim 1 selected from the group consisting of strontium salt of monomethylester of fumaric acid (strontium bis-monomethyl fumarate), strontium salt of monoethylester of fumaric acid (strontium bis-monoethyl fumarate), strontium salt of monopropylester of fumaric acid (strontium bis-monopropyl fumarate), strontium salt of monobutylester of fumaric acid (strontium bis-monobutyl fumarate), and strontium salt of monopentylester of fumaric acid (strontium bis-monopentyl fumarate).

3. The strontium salt according to claim 1, which is a strontium salt of the monomethylester of fumaric acid.

4. The strontium salt according to any one of the claim 1 in combination with one or more selected from the group consisting of di(C1-C5)alkylester of fumaric acid and a mono(C1-C5)alkylester of fumaric acid.

5. The strontium salt according to claim 4 in combination with dimethylfumarate.

6. The strontium salt according to claim 4, in combination with monomethylfumarate, or a pharmaceutically acceptable salt thereof.

7. (canceled)

8. A method for combating tissue degenerative processes comprising administering to a patient in need thereof an effective dosage of a strontium salt according to claim 1.

9. A method for the treatment of one or more of the following conditions selected from the group consisting of: Psoriasis; Psoriatic arthritis; Neurodermatitis; Inflammatory bowel disease, such as Crohn's disease; Ulcerative colitis; autoimmune diseases such as Polyarthritis, Multiple sclerosis (MS), Juvenile-onset diabetes, Hashimoto's thyroiditis, Grave's disease, SLE (systemic lupus erythematosus), Sjögren's syndrome, Pernicious anemia, Chronic active (lupoid) hepatitis, rheumatoid arthritis (RA) and optic neuritis to a patient in need thereof an effective dosage comprising administering a strontium salt according to claim 1.

10. A method for the treatment of one or more conditions, selected from the group consisting of psoriasis, psoriatic arthritis, neurodermatitis and multiple sclerosis (MS) comprising administering to a patient in need thereof an effective dosage of a strontium salt according to claim 1.

11. The method according to claim 10 wherein the condition is psoriasis.

12. A method for the treatment of pain comprising administering to a patient in need thereof an effective dosage of a strontium salt according to claim 1.

13. (canceled)

14. A pharmaceutical composition comprising a strontium salt according to claim 1.

15. A pharmaceutical composition according to claim 14 in the form of a controlled release composition.

16. A method for preparing a strontium salt according to claim 1 the method comprising formation of a dialkyl ester of fumaric acid followed by precipitation by either strontium hydroxide or by strontium chloride in acid solution:

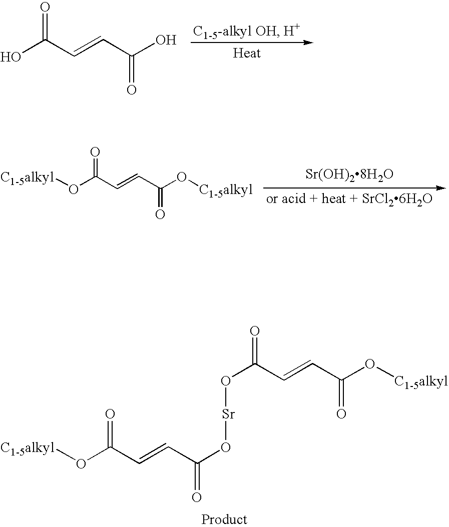

17. A method for preparing a strontium salt according to claim 1 the method comprising formation of fumaric anhydride by a sulphuryl method followed by hydrolysis in the presence of strontium chloride:

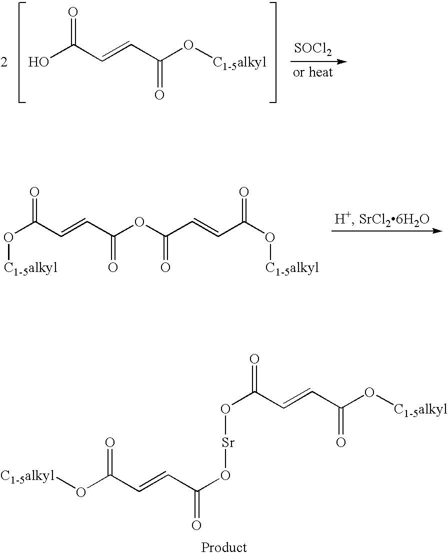

18. A method for preparing a strontium salt according to claim 1, the method comprising reacting the disodium salt of fumaric acid and alkanol in acid solution:

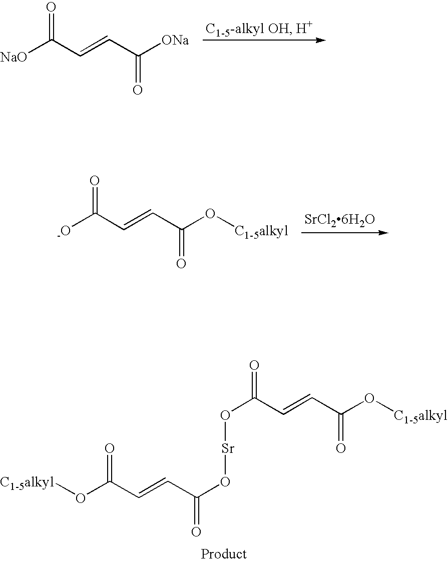

19. A method for preparing a strontium salt according to claim 1 the method comprising formation of bis-monoalkylfumarate from the monoalkyl ester of fumaric acid and precipitation under acid, alkaline or neutral conditions in the presence of strontium chloride:

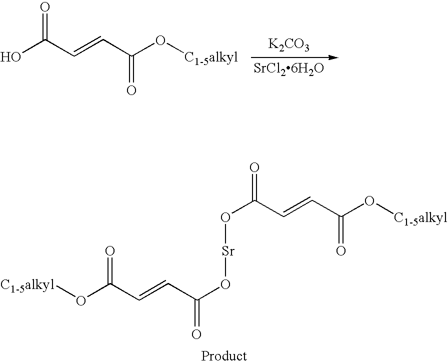

20. A method for preparing a strontium salt according to claim 1 the method comprising reaction of strontium carbonate with monoalkylfumarate: 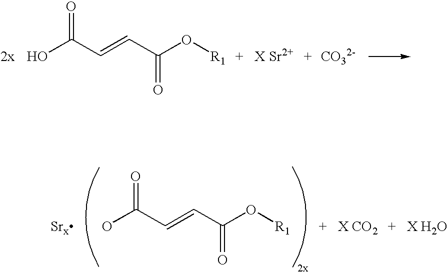
wherein x denotes the stoichiometric factor and R₁ is a (C₁-C₅) alkyl group.

21. A method for preparing a strontium salt as according to claim 1 the method comprising formation of strontium monoalkylfumarate by reaction of strontium hydroxide with monoalkylfumarate dissolved in water.

22. A The strontium salt according to according to claim 1 in crystalline form.

23. The method according to claim 12 wherein the pain is selected from the group consisting of radicular pain, pain associated with radiculopathy, neuropathic pain and sciatica/sciatic pain.

24. A method for the treatment and/or prevention of one or more conditions selected from the group consisting of prevention of rejection following organ transplantation, sarcoidosis, necrobiosis lipoidica, and gramuloma annulare.
